(19) United States
(12) Patent Application Publication
(10) Pub. No.: US 2012/0259210 A1
(43) Pub. Date: Oct. 11, 2012

(54) ULTRASOUND GUIDED POSITIONING OF CARDIAC REPLACEMENT VALVES WITH 3D VISUALIZATION

(76) Inventors: Edward P. Harhen, Duxbury, MA (US); Nicolas Heron, New York, NY (US)

(21) Appl. No.: 13/410,456

(22) Filed: Mar. 2, 2012

Related U.S. Application Data
(60) Provisional application No. 61/474,028, filed on Apr. 11, 2011.
(60) Provisional application No. 61/565,766, filed on Dec. 1, 2011.

(51) Int. Cl. A61B 8/00

(52) U.S. Cl. 600/424

(57) ABSTRACT

A device (e.g., a valve) can be visualized in a patient's body (e.g., in the patient's heart) using an ultrasound system with added position sensors. One position sensor is mounted in the ultrasound probe, and another position sensor is mounted in the device installation apparatus. The device's position with respect to the imaging plane is determined based on the detected positions of the position sensors and known geometric relationships. A representation of the device and the imaging plane, as viewed from a first perspective, is displayed. The perspective is varied to a second perspective, and a representation of the device and the imaging plane, as viewed from the second perspective, is displayed. Displaying the device and the imaging plane from different perspectives helps the user visualize where the device is with respect to the relevant anatomy.

### CROSS REFERENCE TO RELATED APPLICATIONS

This Application claims the benefit of U.S. Provisional Application 61/474,028, filed Apr. 11, 2011, and U.S. Provisional Application 61/565,766, filed Dec. 1, 2011, each of which is incorporated herein by reference.

### BACKGROUND

Conventional percutaneous cardiac valve replacement procedure relies on Trans-Esophageal Echocardiography (TEE) in combination with Fluoroscopy for guiding the valve into position where it is to be deployed. It is easy to see the tissue and the anatomical landmarks on the ultrasound image, but difficult to visualize the valve and its deployment catheter. Conversely, it is easy to see the valve and catheter on the fluoroscopy image, but difficult to clearly see and differentiate the tissue. Since neither imaging modality provides a clear view of both the anatomy and the valve, it difficult to determine exactly where the valve is with respect to the relevant anatomy. This makes positioning of the artificial valve prior to deployment quite challenging.

Relevant background material also includes U.S. Pat. Nos. 4,173,228, 4,431,005, 5,042,486, 5,558,091, and 7,806,829, each of which is incorporated herein by reference.

### SUMMARY OF THE INVENTION

One aspect of the invention is directed to a method of visualizing a device in a patient's body using an ultrasound probe and a device installation apparatus. The ultrasound probe includes an ultrasound transducer that captures images of an imaging plane and a first position sensor mounted so that a geometric relationship between the first position sensor and the ultrasound transducer is known. The device installation apparatus includes the device itself, a device deployment mechanism, and a second position sensor mounted so that a geometric relationship between the second position sensor and the device is known. This method includes the steps of detecting a position of the first position sensor, detecting a position of the second position sensor, and determining a spatial relationship in three-dimensional space between the device and the imaging plane based on (a) the detected position of the first position sensor and the geometric relationship between the first position sensor and the ultrasound transducer and (b) the detected position of the second position sensor and the geometric relationship between the second position sensor and the device. A representation of the device and the imaging plane, as viewed from a first perspective, are displayed, so that a spatial relationship between the representation of the device and the representation of the imaging plane corresponds to the determined spatial relationship. A representation of the device and the imaging plane, as viewed from a second perspective, is also displayed, so that a spatial relationship between the representation of the device and the representation of the imaging plane corresponds to the determined spatial relationship. In some embodiments, the second perspective is displayed after the first perspective. The transition from the first perspective to the second perspective can occur in response to a command received via a user interface. Optionally, a wireframe rectangular parallelepiped (e.g., a cube) with two faces that are parallel to the imaging plane may also be displayed. Optionally, additional perspectives may also be displayed.

Another aspect of the invention is directed to an apparatus for visualizing a position of a device in a patient's body using an ultrasound probe and a device installation apparatus. The ultrasound probe includes an ultrasound transducer that captures images of an imaging plane and a first position sensor mounted so that a geometric relationship between the first position sensor and the ultrasound transducer is known. The device installation apparatus including the device itself, a device deployment mechanism, and a second position sensor mounted so that a geometric relationship between the second position sensor and the device is known. This apparatus includes an ultrasound imaging machine that drives the ultrasound transducer, receives return signals from the ultrasound transducer, converts the received return signals into 2D images of the imaging plane, and displays the 2D images. It also includes a position tracking system that detects a position of the first position sensor, detects a position of the second position sensor, reports the position of the first position sensor to the ultrasound imaging machine, and reports the position of the second position sensor to the ultrasound imaging machine. The ultrasound imaging machine includes a processor that is programmed to determine a spatial relationship in three-dimensional space between the device and the imaging plane based on (a) the detected position of the first position sensor and the geometric relationship between the first position sensor and the ultrasound transducer and (b) the detected position of the second position sensor and the geometric relationship between the second position sensor and the device. The processor is programmed to generate a first representation of the device and a first representation of the imaging plane, as viewed from a first perspective, so that a spatial relationship between the first representation of the device and the first representation of the imaging plane corresponds to the determined spatial relationship. It is also programmed to generate a second representation of the device and a second representation of the imaging plane, as viewed from a second perspective, so that a spatial relationship between the second representation of the device and the second representation of the imaging plane corresponds to the determined spatial relationship. The ultrasound imaging machine displays the first representation of the device and the first representation of the imaging plane, and displays the second representation of the device and the second representation of the imaging plane. In some embodiments, the second representation of the device and the second representation of the imaging plane are displayed after the first representation of the device and the first representation of the imaging plane. In some embodiments, the apparatus may further include a user interface, and a transition from displaying the first representation of the device and the imaging plane to displaying the second representation of the device and the imaging plane may occur in response to a command received via the user interface. Optionally, additional perspectives may be added, and/or a wireframe rectangular parallelepiped with two faces that are parallel to the imaging plane may be displayed together with the device and the imaging plane in each of the different perspectives.

### BRIEF DESCRIPTION OF THE DRAWINGS

FIG. 1 depicts the distal end of an ultrasound probe that includes, in addition to conventional components, a first position sensor.

FIG. 2 depicts the distal end of a valve installation apparatus includes, in addition to conventional components, a second position sensor.

FIG. 3 is a block diagram of a system that makes use of the position sensors to track the position of the valve so that it can be installed at the correct anatomical position.

FIG. 4 depicts the geometric relationship between the ultrasound transducer, the transducer's imaging plane, and two position sensors.

FIG. 5A depicts a wireframe 3D cube that is constructed about a 2D imaging plane, with a representation of the position of the valve when the valve is at a first position.

FIG. 5B depicts the wireframe 3D cube and the 2D imaging plane of FIG. 5A, with a representation of the position of the valve when the valve is at a second position.

FIG. 5C depicts the wireframe 3D cube and the 2D imaging plane of FIG. 5B after being spun to a different perspective.

FIG. 5D depicts the wireframe 3D cube and the 2D imaging plane of FIG. 5B after being tipped to a different perspective.

FIG. 6A depicts an imaging plane at a particular orientation in space.

FIG. 6B depicts how the orientation of a displayed imaging plane is set to match the orientation of the imaging plane in FIG. 6A.

### DESCRIPTION OF THE PREFERRED EMBODIMENTS

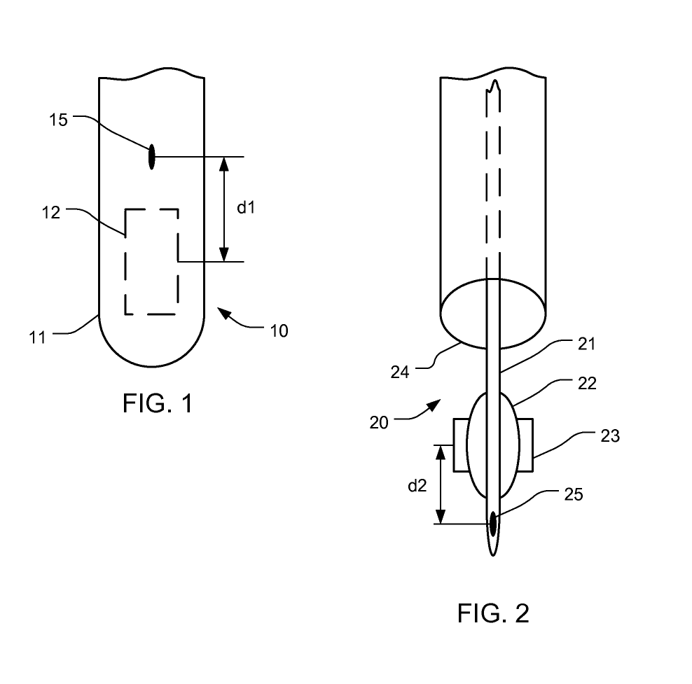

FIGS. 1-4 depict one embodiment of the invention in which the position of the valve may be visualized easily on the ultrasound image so as to make the deployment of the valve much easier due to a much more confident assessment of its position. In this embodiment, position sensors are added to a conventional ultrasound probe and to a conventional valve delivery apparatus, and data from those position sensors is used to determine the location of valve with respect to the relevant anatomy.

FIG. 1 depicts the distal end of an ultrasound probe 10. In most respects, the ultrasound probe 10 is conventional—it has a housing 11 and an ultrasound transducer 12 located within the distal end of the probe 10 and a flexible shaft (not shown). However, in addition to the conventional components, a position sensor 15 is added, together with associated wiring to interface with the position sensor 15. The position sensor 15 can be located anywhere on the distal end of the probe 10, as long as the geometric relationship between the position sensor 15 and the ultrasound transducer 12 is known. Preferably, that relationship is permanently fixed by mounting the ultrasound transducer 12 and the position sensor 15 so that neither can move with respect to the housing 11. Appropriate wiring to the position sensor 15 is provided, which preferably terminates at an appropriate connector (not shown) on the proximal end of the probe. Of course, in alternative embodiments that use a wireless position sensor, the wiring is not necessary.

In the illustrated embodiment, the position sensor is located on the proximal side of the ultrasound transducer 12 by a distance d1 measured from the center of the ultrasound transducer 12 to the center of the position sensor 15. In alternative embodiments, the position sensor 15 can be placed in other locations, such as distally beyond the ultrasound transducer 12, laterally off to the side of the ultrasound transducer 12, or behind the transducer 12. In embodiments that place the position sensor 15 behind the transducer, smaller sensors are preferred to prevent the overall diameter of the ultrasound probe 10 from getting too large.

FIG. 2 depicts the distal end of a valve installation apparatus 20 which is used to deliver a valve 23 to a desired position with respect to a patient's anatomy and then deploy the valve 23 at that position. In most respects, construction of the valve installation apparatus 20 is conventional. A conventional valve 23 is mounted on a conventional deployment mechanism 22 in a conventional manner and delivered through delivery sheath 24, so that once the valve is positioned at the correct location, actuation of the deployment mechanism 22 installs the valve. Examples of suitable valves and valve installation apparatuses include the Sapien Valve System by Edwards Lifesciences, the CoreValve System by Medtronic, and the valve by Direct Flow Medical.

However, in addition to the conventional components described above, a position sensor 25 is added, together with associated wiring to interface with the position sensor 25.

The position sensor 25 is located in a position on the valve installation apparatus 20 that has a known geometric relationship with the valve 23. For example, as shown in FIG. 2, the position sensor 25 can be located on the delivery catheter, at a distance d2 distally or proximal beyond a known position of the valve 23 (measured when the valve is in its undeployed state). Preferably, the valve installation apparatus 20 is constructed so that the spatial relationship will not change until deployment is initiated (e.g., by inflating a balloon). Mechanically adding the position sensor 25 to the valve installation apparatus 20 will depend on the design of the valve installation apparatus 20, and appropriate wiring to the position sensor 25 must be provided, which preferably terminates at an appropriate connector (not shown) on the proximal end of the valve installation apparatus 20. Of course, in alternative embodiments that use a wireless position sensor, the wiring is not necessary.

In alternative embodiments, the position sensor 25 can be placed in other locations, such as on the deployment mechanism 22 or on the delivery sheath 24. In still other alternative embodiments, the position sensor 25 could be positioned on the valve 23 itself (preferably in a way that the position sensor 25 is released when the valve is deployed). However, the position sensor 25 must be positioned so that its relative position with respect to the valve 23 is known (e.g., by placing it at a fixed position with respect to the valve 23). When this is done, it becomes possible to determine the position of the valve 23 by adding an appropriate offset in three dimensional space to the sensed position of the sensor 25.

Commercially available position sensors may be used for the position sensors 15, 25. One example of a suitable sensor is the “model 90” by Ascension Technologies, which are small enough (0.9 mm in diameter) to be integrated into the distal end of the probe 10 and the valve installation apparatus 20. These devices have previously been used for purposes including cardiac electrophysiology mapping and needle biopsy positioning, and they provide six degrees of freedom information (X, Y, and Z Cartesian coordinates) and orientation (azimuth, elevation, and roll) with a high degree of positional accuracy.

Other examples include the sensors made using the technology used by Polhemus Inc. The various commercially available systems differ in the way that they create their signal and perform their signal processing, but at long as they are small enough to fit into the distal end of an ultrasound probe 10 and the valve installation apparatus 20, and can output the appropriate position and orientation information, any technology may be used (e.g., magnetic-based technologies and RF-based systems).

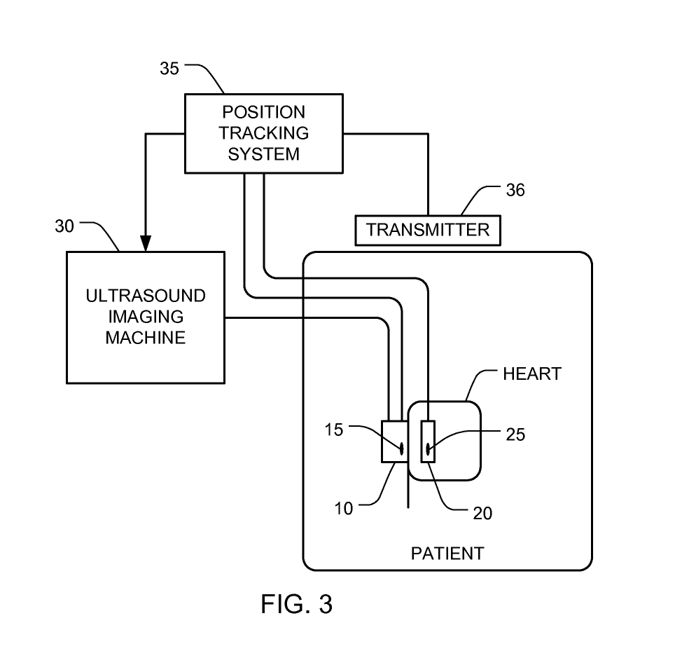

FIG. 3 is a block diagram of a system that makes use of the position sensors 15, 25 to track the position of the valve so that it can be installed at the correct anatomical position. In this system, ultrasound images obtained using the transducer 12 at the distal end of the probe 10 are combined with information obtained by tracking the position sensor 15 on the distal end of an ultrasound probe 10 and the position sensor 25 on the valve installation apparatus 20, to position the valve at a desired spot within the patient's body before deployment.

In FIG. 3, the valve installation apparatus 20 is schematically depicted as being inside the heart of the patient. Access to the heart may be achieved using a conventional procedure (e.g., via a blood vessel like an artery). In addition, FIG. 3, the distal end of the ultrasound probe 10 is shown as being next to the heart. Access to this location is preferably accomplished by positioning the distal end of the probe 10 in the patient's esophagus, (e.g., via the patient's mouth or nose).

The ultrasound imaging machine 30 interacts with the transducer in the distal of the probe 10 to obtain 2D images in a conventional matter (i.e., by driving the ultrasound transducer, receiving return signals from the ultrasound transducer, converting the received return signals into 2D images of the imaging plane, and displaying the 2D images). But in addition to the conventional connection between the ultrasound imaging machine 30 and the transducer in the distal end of the probe 10, there is also wiring between the position tracking system 35 and the position sensor 15 at the distal end of the ultrasound probe. In the embodiment that uses Ascension model 90 position sensors, an Ascension 3D Guidance Medsafe™ electronics unit may be used as the position tracking system 35. Since the wiring between the position tracking system 35 and the position sensor is built into the model 90 sensor, the model 90 sensor may be integrated into the distal end of an ultrasound probe 10 in a way that permits the connector at the proximal end of the model 90 sensor to branch over to the position tracking system 35. In alternative embodiments, the proximal end of the ultrasound probe 10 may be modified so that a single connector that terminates at the ultrasound imaging machine 30 can be used, with appropriate wiring added to route the signals from the position sensor 15 to the position tracking system 35.

A similar position sensor 25 is also disposed at the distal end of the valve installation apparatus 20. A connection between the position sensor 25 and the position tracking system 35 is providing by appropriate wiring that runs from the distal end of the apparatus through the entire length of apparatus and out of the patient's body, and from there to the position tracking system 35. Suitable ways for making the electrical connection between the position tracking system 35 and the position sensor 25 will be apparent to person skilled in the relevant arts. Note that since the distal end of the valve installation apparatus 20 is positioned in the patient's heart during deployment, the wiring must fit within the catheter that delivers the valve installation apparatus 20 to that position, which is typically positioned in the patient's arteries.

With this arrangement, the position tracking system 35 can determine the exact position and orientation in three-dimensional space of the position sensor 15 at the distal end of the ultrasound probe and of the position sensor 25 at the distal end of the valve installation apparatus 20. The position tracking system 35 accomplishes this by communicating with the position sensors 15, 25 via the transmitter 36 which is positioned outside the patient's body, preferably in the vicinity of the patient's heart. This tracking functionality is provided by the manufacturer of the position tracking system 35, and it provides an output to report the position and orientation of the sensors.

A processor (not shown) uses the hardware depicted in FIG. 3 to help guide the valve installation apparatus 20 to a desired position. This processor can be implemented in a stand-alone box, or can be implemented as a separate processor that is housed inside the ultrasound imaging machine 30. In alternative embodiments, an existing processor in the ultrasound imaging machine 30 may be programmed to perform the program steps described herein. But wherever the processor is located, when the distal end of the ultrasound probe 10 is positioned near the patient's heart (e.g., in the patient's esophagus or in the fundus of the patient's stomach), and the distal end of the valve installation apparatus 20 is positioned in the patient's heart in the general vicinity of its target destination, the system depicted in FIG. 3 can be used to accurately position the valve 23 at a desired location by performing the steps described below.

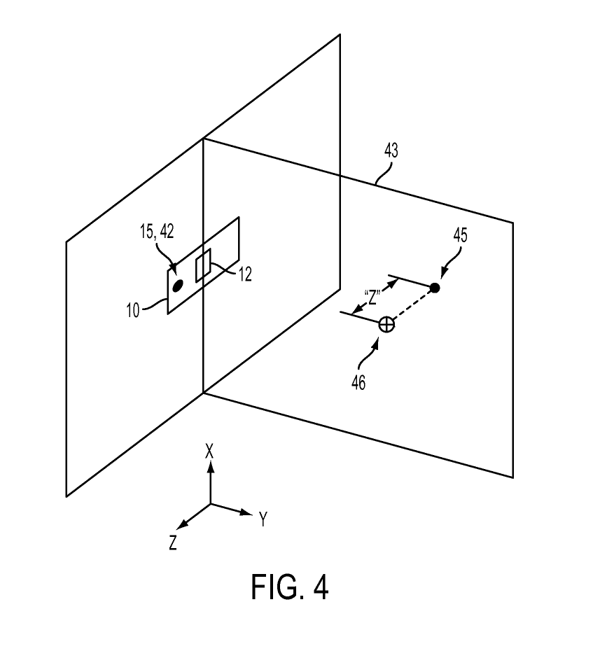

Referring now to FIGS. 1-4, taken together, the position tracking system 35 first reports the location and orientation of the position sensor 15 to the processor. That position is depicted as point 42 in FIG. 4. Because of the fixed geometric relationship between the position sensor 15 and the ultrasound transducer 12, and the known relationship between the ultrasound transducer 12 and the imaging plane 43 of that transducer, the processor can determine the location of the imaging plane 43 (referred to herein as the XY plane) in space based on the sensed position and orientation of the position sensor 15.

The position tracking system 35 also determines the position of the position sensor 25 at the distal end of the valve installation apparatus 20. That position is depicted as point 45 in FIG. 4. Then, based on the known location of point 45 and the known location of the XY plane 43 (which was calculated from the measured position 42 and the known offset between point 42 and the ultrasound transducer 12), the processor computes a projection of point 45 onto the XY plane 43 and the distance Z between point 45 and the XY plane. This projection is labeled 46 in FIG. 4.

The processor then sends the signed value of Z and the coordinates of point 46 to the software object in the ultrasound imaging machine 30 that is responsible for generating the images that are ultimately displayed. That software object is modified with respect to conventional ultrasound imaging software so as to display the location of point 46 on the ultrasound image. This can be accomplished, for example, by displaying a colored dot at the position of point 46 on the XY plane 43. The modifications that are needed to add a colored dot to an image generated by a software object will be readily apparent to persons skilled in the relevant arts.

Preferably, the distance Z is also displayed by the ultrasound imaging machine 30. This can be accomplished using any of a variety of user interface techniques, including but not limited to displaying a numeric indicator of the value of Z to specify the distance in front of or behind the XY imaging plane 43, or displaying a bar graph whose length is proportional to the distance Z and whose direction denotes the sign of Z. In alternative embodiments other user interface techniques may be used, such as relying on color and/or intensity to convey the sign and magnitude of Z to the operator. The modifications that are needed to add this Z information to the ultrasound display will also be readily apparent to persons skilled in the relevant arts.

When the system is configured in this way, during use the operator will be able to see the relevant anatomy by looking at the image that is generated by the ultrasound imaging machine 30. Based on the position of the dot representing point 46 that was superposed on the imaging plane, and the indication of the value of Z, the operator can determine where the position sensor 25 is with respect to the portion of the patient's anatomy that appears on the display of the ultrasound imaging machine 30.

Based on the known geometric offset between the position sensor 25 and the valve 23, the operator can use the image displayed by the ultrasound imaging machine 30, the position point 46 that is superposed on that image, and the display of Z information to position the valve at the appropriate anatomical location.

In alternative preferred embodiments, instead of having the operator account for the offset between the position sensor 25 and the valve 23, the system is programmed to automatically offset the displayed value of the Z by the distance d2, which eliminates the need for the operator to account for that offset himself. In these embodiments, the procedure of valve deployment becomes very simple. The valve installation apparatus 20 is snaked along the blood vessel until it is in the general vicinity of the desired position. Then, the operator aligns the imaging plane with the a cross sectional view of the desired position within the patients original valve that is being treated by, for example, advancing or retracting the distal end of an ultrasound probe 10, and/or flexing a bending section of that probe. An indication that the proper position has been reached is when (a) the imaging plane displayed on the ultrasound imaging machine 30 depicts the desired position within the patients original valve, (b) the position marker 46 that is superposed on the ultrasound image indicates that the valve is aligned within the desired position of the valve, and (c) the Z display indicates that Z=0. After this, the deployment mechanism 22 can be triggered (e.g., by inflating a balloon), which deploys the valve.

In the above-described embodiments, the information is presented to the user in the form of a conventional 2D ultrasound image with (1) a position marker added to the image plane to indicate a projection of the valve's location onto the image plane and (2) and indication of the distance between the valve and the image plane. In alternative embodiments, different ways to help the user visualize the position of the valve with respect to the relevant anatomy may be used.

One such approach is to make a computer-generated model of an object in 3D space, in which the object incorporates both the valve and the 2D imaging plane that is currently being imaged by the ultrasound system. Using a suitable user interface, the user can then view the object from different perspectives using 3D image manipulation techniques that are commonly used in the context of computer aided design (CAD) systems and gaming systems. A suitable user interface, which can be implemented using any of a variety of techniques used in conventional CAD and gaming systems, then enables the user to view the object from different perspectives (e.g., by rotating the object about horizontal and/or vertical axes).

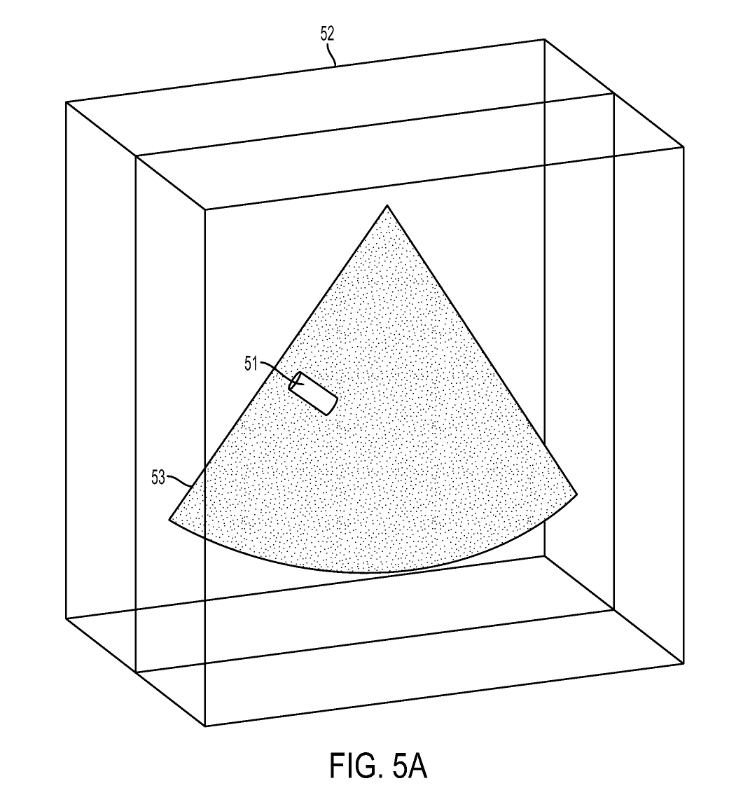

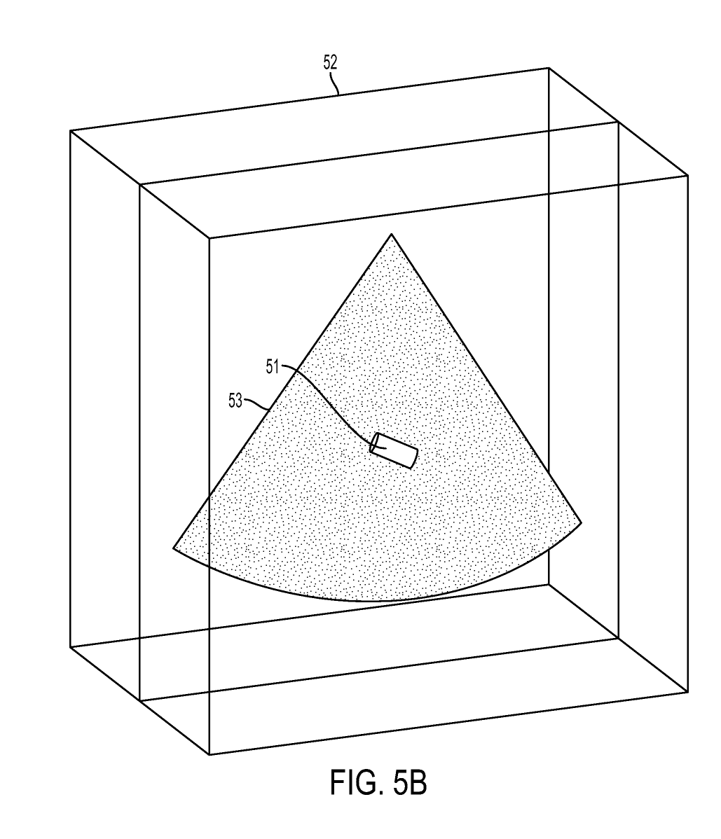

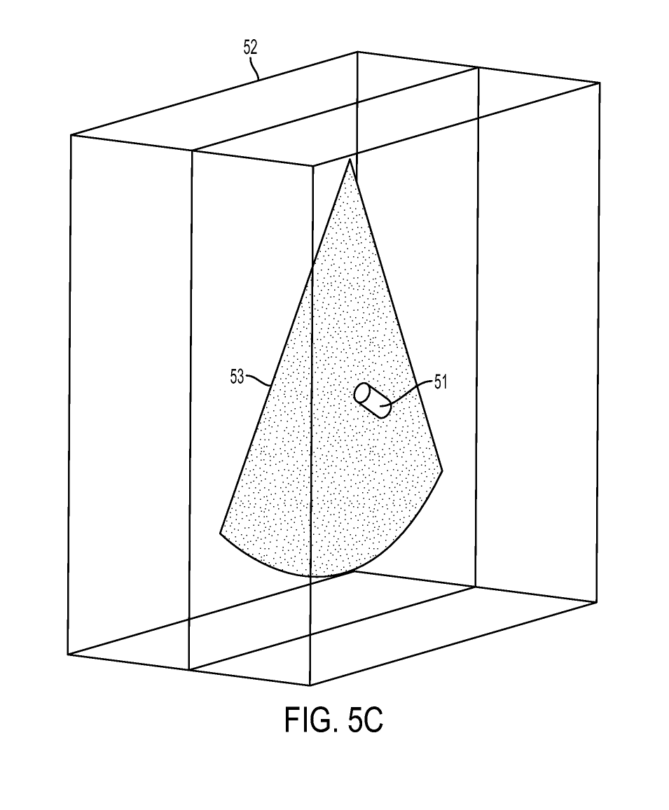

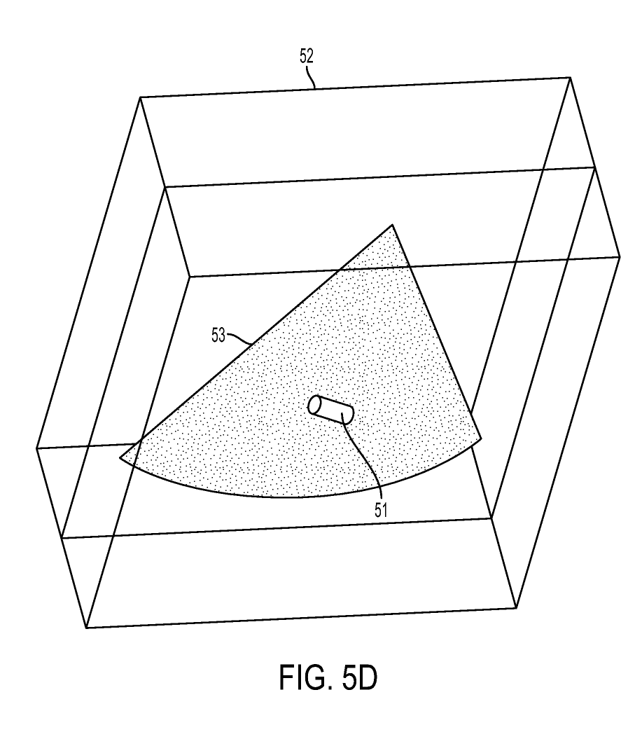

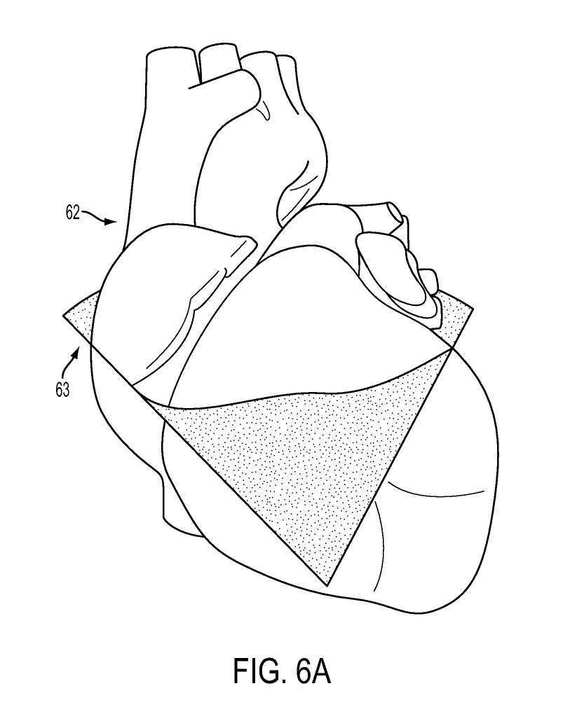

FIG. 5A depicts such an object in 3D space, and the object has three components: a wireframe 3D cube 52, the 2D imaging plane 53 that is currently being imaged by the ultrasound system, and a cylinder 51 that represents the position of the position sensor 25 (shown in FIG. 2). The starting frame of reference for creating the object is the imaging plane 53, whose position in space (with respect to the ultrasound transducer) is known based on the fixed geometric relationship between the ultrasound transducer 12 and the position sensor 15 (both shown in FIG. 2), and the detected position of the position sensor, as described above. The system then adds the wire frame cube 52 at a location in space that positions both the front and rear faces of the wire frame cube 52 parallel to the imaging plane 53, preferably with the imaging plane 53 at the median plane of the 3D cube. The system also adds the cylinder 51 to the object at an appropriate location that corresponds to the detected position of position sensor 25 (shown in FIG. 2). Preferably, the spatial relationship in three-dimensional space between the cylinder and the imaging plane is determined based on (a) the detected position of the first position sensor and the geometric relationship between the first position sensor and the ultrasound transducer and (b) the detected position of the second position sensor and the geometric relationship between the second position sensor and the device, as explained above. In alternative embodiments, the cube may be omitted, and in other embodiments, a rectangular parallelepiped or another geometric shape may be used instead of a cube.

Since the valve is in a fixed geometric relationship with the position sensor 25, moving the valve to a new position is detected by the system, and the system responds to the detected movement by moving the cylinder 51 to a new position within the 3D object, as shown in FIG. 5B. Preferably, the object can be rotated by the user to help the user better visualize the location of the position sensor 25 in 3D space. Assume, for example, that the position sensor 25 remains at the location that caused the system to paint the cylinder 51 at the location shown in FIG. 5B, as viewed from a first perspective. Initially, the display that is presented to the user includes a first representation of the device and a first representation of the imaging plane, as viewed from the first perspective, so that a spatial relationship between the first representation of the device and the first representation of the imaging plane corresponds to the spatial relationship determined based on measurements from the position sensors and subsequent computations.

If the user wants to view the geometry from a different perspective, he can use the user interface to spin the perspective to a second view shown in FIG. 5C, or to tip the perspective to a third view shown in FIG. 5D. The second and third views both include representations of the device and the imaging plane, as viewed from second and third perspective, respectively, so that a spatial relationship between the device and the imaging plane corresponds to the spatial relationship determined based on measurements from the position sensors and subsequent computations.

Other 3D operations (e.g., translations, rotations, and zooming) can be implemented as well. The display of a 2D image as a slice within the 3D wireframe enhances the perception of the position sensor 25 relative to the imaging plane. Implementing the rotation of the object may be handled by conventional video hardware and software. For example, when a 3D object is created in memory in a conventional video card, the object can be moved and rotated by sending commands to the video card. A suitable user interface and software can then be used to map the user's desired viewing perspective into those commands.

In alternative embodiments, instead of having the cylinder 51 represent the position of the position sensor, the cylinder 51 can be used to represent the position of the valve that is being deployed. In these embodiments, the cylinder would be painted onto the object at a location that is offset from the location of the position sensor 25 based on the known geometric relationship between the valve and the position sensor 25. Optionally, instead of using a plain cylinder 51 in these embodiments, a more accurate representation of the shape of the undeployed valve can be displayed at the appropriate position within the 3D object.

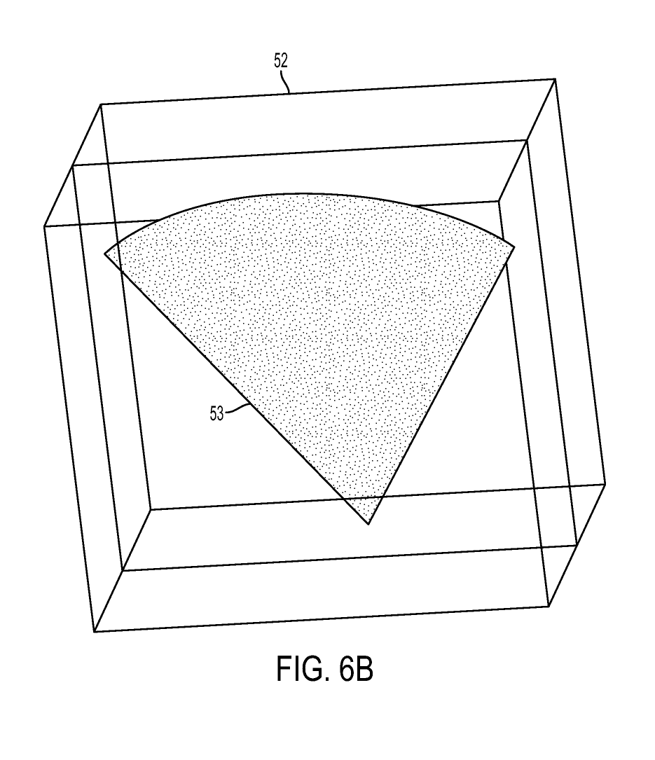

Optionally, the system may be programmed to display the object in an anatomic orientation upon request from the user (e.g., in response to a request received via a user interface), which would show the imaging plane at the same orientation in which imaging plane is physically oriented in 3D space. For example, assuming the patient is lying down and the ultrasound transducer is used to image the patient's heart 62, if the imaging plane 63 of the ultrasound transducer is canted by about 30°, and spun by an angle of about 10°, as shown in FIG. 6A, the display that is presented to the user would be set up to match those angles, as shown in FIG. 6B. In this mode, the orientation of the displayed imaging plane 53 is preferably set to automatically follow changes in the transducer's orientation based on the position and orientation information of the position sensor 15 that is built into the ultrasound probe 10 (shown in FIG. 1).

Optionally, proximity of the ultrasound imaging plane 53 can be indicated by modifying the color and/or size of the rendered cylinder, adding graphics onto or in proximity of the sensor display (e.g., a circle with a radius that varies proportionally with the distance between the sensor and the imaging plane), or a variety of alternative approaches (including but not limited to numerically displaying the actual distance).

Optionally, the techniques described above can be combined with conventional fluoroscopic images, which may be able to provide additional information to the operator, or as a double-check that the valve is properly positioned.

The techniques described above advantageously help determine the position of the valve relative to the tissue being visualized in the imaging plane, and improve the confidence of the correct placement of the valve when deployed. The procedures can also eliminate or at least reduce the amount of fluoroscopy or other x-ray based techniques, advantageously reducing the physician's and patient's exposure to same.

The concepts discussed above can be used with any type of ultrasound probe that generates an image, such as Trans-Esophageal Echocardiography probes (e.g., those described in U.S. Pat. No. 7,717,850, which is incorporated herein by reference), Intracardiac Echocardiography Catheters (e.g., St. Jude Medical's ViewFlex™ PLUS ICE Catheter and Boston Scientific's Ultra ICE™ Catheter), and other types of ultrasound imaging devices. The concepts discussed above can even be used with imaging modalities other than ultrasound, such as MRI and CT devices. In all these situations, one position sensor is affixed to an imaging head in a fixed relationship with an image plane, and another position sensor is affixed to the prosthesis or other the medical device that is being guided to a position in the patient's body. The fixed relationship between the position sensor and the image plane can be used as described above to help guide the device into the desired position.

Note that while the invention is described above in the context of installing heart valves, it can also be used to help position other devices at the correct locations in a patient's body. It could even be used in non-medical contexts (e.g., guiding a component to a desired position within a machine that is being assembled).

Finally, while the present invention has been disclosed with reference to certain embodiments, numerous modifications, alterations, and changes to the described embodiments are possible without departing from the sphere and scope of the present invention.

We claim:

1. A method of visualizing a device in a patient's body using an ultrasound probe and a device installation apparatus, the ultrasound probe including an ultrasound transducer that captures images of an imaging plane and a first position sensor mounted so that a geometric relationship between the first position sensor and the ultrasound transducer is known, the device installation apparatus including the device, a device deployment mechanism, and a second position sensor mounted so that a geometric relationship between the second position sensor and the device is known, the method comprising the steps of:
detecting a position of the first position sensor;
detecting a position of the second position sensor;
determining a spatial relationship in three-dimensional space between the device and the imaging plane based on (a) the detected position of the first position sensor and the geometric relationship between the first position sensor and the ultrasound transducer and (b) the detected position of the second position sensor and the geometric relationship between the second position sensor and the device;
a first displaying step that includes displaying a first representation of the device and a first representation of the imaging plane, as viewed from a first perspective, so that a spatial relationship between the first representation of the device and the first representation of the imaging plane corresponds to the spatial relationship determined in the determining step; and
a second displaying step that includes displaying a second representation of the device and a second representation of the imaging plane, as viewed from a second perspective, so that a spatial relationship between the second representation of the device and the second representation of the imaging plane corresponds to the spatial relationship determined in the determining step.

2. The method of claim 1, wherein the second displaying step occurs later in time than the first displaying step.

3. The method of claim 2, wherein a transition from the first displaying step to the second displaying step occurs in response to a command received via a user interface.

4. The method of claim 1, wherein the first displaying step further includes displaying a wireframe rectangular parallelepiped with two faces that are parallel to the imaging plane, as viewed from the first perspective, and wherein the second displaying step further includes displaying the parallelepiped as viewed from the second perspective.

5. The method of claim 4, wherein the parallelepiped is a cube and the two faces of the parallelepiped that are parallel to the imaging plane are equidistant from the imaging plane.

6. The method of claim 1, wherein the second displaying step occurs later in time than the first displaying step, wherein a transition from the first displaying step to the second displaying step occurs in response to a command received via a user interface, wherein the first displaying step further includes displaying a wireframe rectangular parallelepiped with two faces that are parallel to the imaging plane, as viewed from the first perspective, wherein the second displaying step further includes displaying the parallelepiped as viewed from the second perspective, wherein the first displaying step comprises sending signals to a two-dimensional display, and wherein the second displaying step comprises sending signals to the two-dimensional display.

7. The method of claim 6, further comprising a third displaying step that includes displaying a third representation of the device and a third representation of the imaging plane, as viewed from a third perspective, so that a spatial relationship between the third representation of the device and the third representation of the imaging plane corresponds to the spatial relationship determined in the determining step, wherein the third displaying step occurs later in time than the second displaying step, and wherein a transition from the second displaying step to the third displaying step occurs in response to a command received via the user interface.

8. The method of claim 1, wherein the first displaying step comprises sending signals to a two-dimensional display, and wherein the second displaying step comprises sending signals to the two-dimensional display.

9. The method of claim 1, wherein the device comprises a valve, the device installation apparatus comprises a valve installation apparatus, and the device deployment mechanism comprises a valve deployment mechanism.

10. An apparatus for visualizing a position of a device in a patient's body using an ultrasound probe and a device installation apparatus, the ultrasound probe including an ultrasound transducer that captures images of an imaging plane and a first position sensor mounted so that a geometric relationship between the first position sensor and the ultrasound transducer is known, the device installation apparatus including the device, a device deployment mechanism, and a second position sensor mounted so that a geometric relationship between the second position sensor and the device is known, the apparatus comprising:
an ultrasound imaging machine that drives the ultrasound transducer, receives return signals from the ultrasound transducer, converts the received return signals into 2D images of the imaging plane, and displays the 2D images; and
a position tracking system that detects a position of the first position sensor, detects a position of the second position sensor, reports the position of the first position sensor to the ultrasound imaging machine, and reports the position of the second position sensor to the ultrasound imaging machine,
wherein the ultrasound imaging machine includes a processor that is programmed to determine a spatial relationship in three-dimensional space between the device and the imaging plane based on (a) the detected position of the first position sensor and the geometric relationship between the first position sensor and the ultrasound transducer and (b) the detected position of the second position sensor and the geometric relationship between the second position sensor and the device, and wherein the processor is programmed to (i) generate a first representation of the device and a first representation of the imaging plane, as viewed from a first perspective, so that a spatial relationship between the first representation of the device and the first representation of the imaging plane corresponds to the determined spatial relationship, and (ii) generate a second representation of the device and a second representation of the imaging plane, as viewed from a second perspective, so that a spatial relationship between the second representation of the device and the second representation of the imaging plane corresponds to the determined spatial relationship, and
wherein the ultrasound imaging machine displays the first representation of the device and the first representation of the imaging plane, and displays the second representation of the device and the second representation of the imaging plane.

11. The apparatus of claim 10, wherein the ultrasound imaging machine displays the second representation of the device and the second representation of the imaging plane after displaying the first representation of the device and the first representation of the imaging plane.

12. The apparatus of claim 11, wherein the apparatus further comprises a user interface, and a transition from displaying the first representation of the device and the first representation of the imaging plane to displaying the second representation of the device and the second representation of the imaging plane occurs in response to a command received via the user interface.

13. The apparatus of claim 12, wherein the processor is further programmed to generate a third representation of the device and a third representation of the imaging plane, as viewed from a third perspective, so that a spatial relationship between the third representation of the device and the third representation of the imaging plane corresponds to the determined spatial relationship,
wherein the ultrasound imaging machine displays the third representation of the device and the third representation of the imaging plane, and
wherein a transition from displaying the second representation of the device and the second representation of the imaging plane to displaying the third representation of the device and the third representation of the imaging plane occurs in response to a command received via the user interface.

14. The apparatus of claim 10, wherein processor is further programmed to execute the steps of generating a model of a wireframe rectangular parallelepiped with two faces that are parallel to the imaging plane, determining how the model would look when viewed from the first perspective, and determining how the model would look when viewed from the second perspective, and
wherein the ultrasound imaging machine displays how the model would look when viewed from the first perspective and displays how the model would look when viewed from the second perspective.

15. The apparatus of claim 14, wherein the parallelepiped is a cube and the two faces of the parallelepiped that are parallel to the imaging plane are equidistant from the imaging plane.

16. The apparatus of claim 10, wherein the apparatus further comprises a user interface that accepts commands from a user to rotate a viewing perspective.

17. The apparatus of claim 10, wherein the device comprises a valve, the device installation apparatus comprises a valve installation apparatus, and the device deployment mechanism comprises a valve deployment mechanism.
